(19) United States
(12) Patent Application Publication
(10) Pub. No.: US 2008/0045980 A1
(43) Pub. Date: Feb. 21, 2008

(54) Surgical Device for Guiding at Least Two Suture Threads Through the Edge Region of a Tissue Opening

(76) Inventor: Werner Schwarz, Ruhpolding (DE)

Correspondence Address: WILLIAM COLLARD;COLLARD & ROE, P.C., 1077 NORTHERN BOULEVARD, ROSLYN, NY, 11576, US

(21) Appl. No.: 11/660,616

(22) PCT Filed: Aug. 25, 2005
(86) PCT No.: PCT/DE05/01497

§ 371 Date: Feb. 20, 2007

(30) Foreign Application Priority Data
Aug. 30, 2004 (DE) 10 2004 041 936.1

(51) Int. Cl. A61B 17/04

(52) U.S. Cl. 606/148

(57) ABSTRACT

The invention relates to a surgical device for guiding at least two sewing threads through the marginal area of a tissue opening which is present especially in an artery of an individual, the opening optionally having been produced by cutting. The device comprises a bar-shaped needle and thread guiding device having a needle transfer area at the proximal end of the device, a needle receiving area at the distal end of the device, and arranged thereinbetween an intermediate area for receiving the marginal area of the tissue opening. The needle transfer area, the intermediate area and the needle receiving area are rigidly interlinked. The bar-shaped needle and thread guiding device can be closed by an elongate outer member which positively surrounds the intermediate area. The outer member can be displaced in relation to the bar-shaped needle and thread guiding device in the longitudinal direction thereof, thereby releasing the intermediate area. The needle transfer area, towards the distal end of the device, and the elongate member, in the area towards the proximal end of the device, have end faces facing each other.

The invention relates to a surgical device for guiding at least two suture threads through the edge region of a tissue opening, particularly one present in the artery of an individual, if applicable formed by an incision, and for retracting the suture thread ends guided through the edge region in question, in each instance, from the stated opening, having a rod-shaped needle and thread guidance device, in which suture threads attached to needles, in each instance, are guided in guidance and/or accommodation openings, in such a manner that these can be moved from a needle dispensing region disposed at the proximal end of the device to a needle accommodation region at the distal end of the device, by way of an intermediate space region, whereby the intermediate space region in question has a length that at least corresponds to the thickness of the wall of the tissue opening, and an outer shape, with reference to the outer shape of the needle dispensing region and/or the needle accommodation region, that is reduced for unhindered movement of the needles in question between the needle regions in question, and whereby the stated needles can be moved to the needle accommodation region, for permanent accommodation there, after having been guided through the edge region of the tissue opening in question, and whereby afterwards, the rod-shaped needle and thread guidance device, together with the needles permanently accommodated in the needle accommodation region, and the suture thread ends connected with them, can be retracted from the stated tissue opening again, in such a manner that the latter can be closed by means of drawing together all of the suture thread ends, outside of the tissue opening in question.

A device of the type stated above is already known (DE 199 42 951 C1=EP 1 217 955). In the case of this known device, the rod-shaped needle and thread guidance device is formed by means of three parts that can be rotated relative to one another, specifically by a thread feed device situated at the proximal end of the device, a thread accommodation part provided at the distal end of the device, and a central thread release/thread clamping part that lies between them. In this connection, the central thread release/thread clamping part in question has such a cross-section that it allows introduction of the suture threads fed in at the proximal end of the device into exposed accommodation openings provided in the thread accommodation part at the distal end of the device, in at least one rotational position, and makes it possible to hold the suture threads accommodated in the accommodation openings in question, along with the needles, tightly in a rotational position that is different from this rotational position, for pulling the entire thread guidance device out of the stated opening. Although openings in a wall of a sheath, particularly in an artery wall of an individual, can be satisfactorily closed with this known device, there is the desire to make available a surgical device that is simpler to operate, for guiding at least two suture threads through the edge region of a tissue opening, in the case of which it is possible to do without rotational movement of individual parts of the device.

A suture device is already known (WO 94/08516), in which up to four needles connected with suture threads can be guided through a wall of a blood vessel, close to the edge region of an opening situated in it, by means of a pushing device, and accommodated by an accommodation device situated within the blood vessel in question, in the region of the stated opening. This accommodation device is formed by means of a needle accommodation device in the form of a catch basket, which at first is guided through the opening in question, in the collapsed state, and then is opened up in the opening in question, in order to thereupon lie against the edge region of the opening in question, within the blood vessel. The needles guided through the edge region of the aforementioned opening can be accommodated by this needle accommodation device in the form of a catch basket, which device is subsequently rotated about its longitudinal axis, in order to hold the needles in question tightly. Afterwards, the relevant needle accommodation device in the form of a catch basket is collapsed, together with the needles contained in it, in order to be completely pulled out of the aforementioned opening. In this procedure, the suture threads connected with the needle ends are drawn out of supply magazines, drawn through the puncture sites of the aforementioned needles, close to the edge region of the aforementioned opening, and drawn out of the opening in question again. Outside of the aforementioned opening, the suture thread ends can then be drawn together and knotted, if necessary, in order to close the opening in question.

Although the known device considered above has a relatively simple structure in terms of its principle, problems can occur during the accommodation and holding of the needles guided through the edge region of the opening, in each instance, in the needle accommodation device in the form of a catch basket, if one or more needles cannot be held tightly by this needle accommodation device. In such cases, complicated interventions are then required, in order to remove the needles contained in the blood vessel.

Furthermore, a device for guiding two suture threads through a wall of a blood vessel, close to the edge region of an opening formed in it, is known (WO 94/13211), which contains a needle carrier to be introduced into the opening in question, and a needle accommodation device situated outside the opening in question. The needle carrier is equipped with suture threads, the ends of which are connected with needles. In use, the aforementioned needle carrier is first completely introduced into the blood vessel to be closed, through the aforementioned opening, and subsequently the needles are guided through the edge region of the opening in question, from the inside to the outside. In this connection, the remaining needle carrier parts remain in the blood vessel at first. Subsequently, these needle carrier parts are pulled out of the blood vessel, through the aforementioned opening, so that only the suture thread connected with the needles that have already been guided out remains in the blood vessel in question. By means of further retracting the needles, the suture thread in question is finally tensed in the interior of the blood vessel in question, so that afterwards, the suture thread ends can be knotted. In this way, the opening in question is then closed.

The known device just considered does permit guiding suture threads through the wall of a blood vessel, close to the edge region of an opening present in it, in terms of principle; however, safety in connection with the accommodation of the needle ends in the needle accommodation device is at least critical. This is because if one needle or another is not securely accommodated by the needle accommodation device, additional interventions are also required in this case, in order to avoid complications. For reasons of dimensions, the access to the blood vessel, specifically and in particular to the artery, must be widened. As a result, the known device in question is therefore not minimally invasive.

Furthermore, a device for guiding at least one suture thread through the wall of a blood vessel of an individual, close to the edge region of an opening present in it, is known (WO 95/13021, U.S. Pat. No. 5,527,322, U.S. Pat. No. 5,792,152). This known device has a rod-shaped thread guidance device, at the tip of which a nose part having a needle deflection track is provided, which is connected with the rod-shaped thread guidance device by way of a region having a reduced cross-section. In this rod-shaped thread guidance device, there is a needle feed opening, which is oriented towards the entry side of the needle deflection track. Furthermore, there is a second needle guidance opening in the rod-shaped thread guidance device, which is oriented towards the exit side of the needle deflection track in the nose part. Because of this structure, the known device just considered functions as follows. First, the entire device is introduced into the opening of a blood vessel of an individual that is to be closed, to such an extent that the wall of the opening in question lies against the aforementioned region having a reduced cross-section, by way of which the nose part is connected with the rod-shaped thread guidance device. However, secure and precise positioning of the device in question in the edge region of the blood vessel to be closed is not possible here, because of the aforementioned region having a reduced cross-section. Then, a needle connected with a suture thread is moved forward towards the nose part, through the needle feed opening of the rod-shaped thread guidance device, whereby the needle in question penetrates the wall of the blood vessel close to the edge region of the aforementioned opening, in this connection, and subsequently is deflected in the deflection track of the nose part, in such a manner that it then penetrates the edge region of the vessel wall from the inside to the outside. Subsequently, the needle in question is guided back through the aforementioned further needle guidance opening of the rod-shaped thread guidance device, so that the edge region of the aforementioned opening now has a thread pulled through it at two sites that lie diametrically opposite one another. This thread must subsequently be guided out of the deflection track, by way of a thread release slit arrangement, so that thereupon, the entire device can be pulled out of the opening of the aforementioned blood vessel.

It is true that the known device considered above permits guiding at least one suture thread through the wall of a blood vessel, close to the edge region of an opening present in it, at two diametrically opposite sites, in terms of principle; however, in practice the aforementioned needle deflection design sometimes presents problems, since only flexible needles or small needles can be used due to the relatively great curvature of the deflection track present in the aforementioned nose part, and this can cause problems when guiding such needles through vessel walls.

Finally, a suture device for guiding the ends of a suture thread through the wall of a blood vessel of an individual close to the edge region of an opening present in it is also known (U.S. Pat. No. 5,860,990). In the case of this known device, a suture thread supply with loop-shaped thread ends is introduced into the blood vessel through the opening in question, by means of a rod-shaped thread feed device. Outside of the rod-shaped thread supply device in question, needle-shaped thread accommodators are provided at two diametrically opposite sites, which are supposed to accommodate the thread ends configured in loop shape after the vessel wall has been punctured, close to the edge region of the opening in question, and then to pull them outward, out of the blood vessel.

Although the known device last considered makes it possible to guide ends of a suture thread configured in loop shape through a blood vessel wall, close to the edge region of an opening present in it, in terms of principle, this design also demonstrates problems with the accommodation of the aforementioned loop-shaped thread ends during its use. This is because secure accommodation of the loop-shaped thread ends in question is only guaranteed if the thread ends in question are held tightly by the rod-shaped thread feed device, in such a defined position that the needle-shaped thread accommodators can actually grasp these loop-shaped thread ends. In practice, this can sometimes be achieved only with significant difficulties.

The invention is based on the task of further developing a surgical device of the type stated initially, in such a manner that it is possible to do without rotating elements when guiding at least two suture threads through the edge region of a tissue opening, and, at the same time, secure positioning of the device in question in the tissue opening in question is made possible.

The task indicated above is accomplished, according to the invention, in the case of a device of the type stated initially, in that the needle dispensing region, the intermediate space region, and the needle accommodation region of the rod-shaped needle and thread guidance device are rigidly connected with one another,

that the rod-shaped needle and thread guidance device can be closed off by an elongated outer member that surrounds the intermediate space region with shape fit, which member can be displaced relative to the rod-shaped thread guidance device, in its longitudinal direction, releasing the stated intermediate space region,

and that the needle dispensing region has an end side towards the distal end of the device, and the elongated outer member has an end side in its region towards the proximal end of the device, which end sides face one another.

The invention is characterized, as compared with the previously known devices for closing a tissue opening of an individual, by the advantage that secure and precise positioning of the device in question in the tissue opening to be closed by means of at least two suture threads is made possible, with particularly little effort, in total, without rotating elements being required in this connection. This is because according to the present invention, only a rigid rod-shaped needle and thread guidance device, in other words one having no rotating elements, is provided, with which the desired secure and precise positioning of the device in the tissue opening to be closed can be achieved, taking advantage of the distal end of the needle dispensing region and the edge region of the elongated outer member that is present, facing the proximal end of the device, when this outer member is moved towards the distal end of the device, releasing the intermediate space region between the needle dispensing region and the needle accommodation region of the device according to the invention. In this case, the edge region of the tissue opening to be closed by means of suture threads is clamped into the aforementioned intermediate space region of the device according to the invention, between its thread dispensing region and thread accommodation region, whereby the site at which the needles with the suture thread ends attached to them can subsequently be guided through the aforementioned edge region of the tissue opening is precisely and securely established.

It is practical if the end sides of the needle dispensing region on the distal end of the device and of the elongated outer member in its region towards the proximal end of the device run parallel to one another, and preferably the end sides in question run at a right angle to the longitudinal direction of the rod-shaped thread guidance device. As a result, the end sides in question lie against the two sides of the edge region of the tissue opening to be closed, in particularly simple and reliable manner.

It is furthermore advantageous if, according to another embodiment of the invention, the needle accommodation openings of the needle accommodation region are configured in funnel shape in their needle inlet region, in each instance. This facilitates the introduction of the needles into the needle accommodation openings in question, in advantageous manner.

A particularly advantageous embodiment of the device according to the invention results if the needle accommodation region has a bevel that runs from its outside circumference in the direction towards the center axis of the device, on its side facing the proximal end of the device, which bevel preferably forms an angle of ≦45° with reference to the center axis of the device. By means of this measure, it is possible to easily pull the device according to the invention out of the tissue opening to be closed, in each instance, after the elongated outer member, which at first closed off the intermediate space region between the needle dispensing region and the needle accommodation region of the needle and thread guidance device, is moved towards the distal end of the device, into a position that completely releases the intermediate space in question.

It is practical if the rod-shaped needle and thread guidance device has a passage opening in its intermediate space region, which opening is connected with a connection opening present in the rod-shaped needle and thread guidance device that runs towards its proximal end. By means of this measure, it can be additionally determined, in particularly simple manner, when the device according to the invention is correctly positioned in the tissue opening to be closed, in each instance. This is because when the device in question is correctly positioned, tissue fluid contained in the tissue opening, such as blood, can get through the aforementioned passage opening and the connection opening connected with it to the proximal end of the device, and thereby provide an additional optical indication for correct positioning of the entire device.

It is practical if the elongated outer member also has a passage opening, which is connected with the passage opening situated in the intermediate space region of the rod-shaped needle and thread guidance device in the position of the elongated outer member that closes off the aforementioned intermediate space region of the rod-shaped needle and thread guidance device. In this way, the specific site at which the intermediate space region in question is able to accommodate the edge region of the tissue opening in question can be determined in particularly simple manner, while the intermediate space region of the rod-shaped needle and thread guidance device is completely closed off by the elongated outer member, while the device is being introduced into the tissue opening to be closed off, in each instance, if subsequently the aforementioned elongated outer member is displaced towards the distal end of the device, in order to release the intermediate space region.

It is practical if the rod-shaped needle and thread guidance device has an oval cross-section, and the needle dispensing region and the needle accommodation region are disposed close to the end regions only on the larger cross-section axis of the oval-shaped cross-section in question, in this connection. In this manner, a relatively low stress on the tissue opening to be closed, in each instance, due to its widening in cross-section after introduction of the rod-shaped needle and thread guidance device, is assured, in advantageous manner.

According to another practical further development of the present invention, however, the rod-shaped needle and thread guidance device can also have a circular cross-section or a cross-section that approximates the latter, and the needle dispensing region and the needle accommodation region can be disposed distributed around the cross-section in question, with corresponding needle guidance openings or needle accommodation openings, respectively. This makes it possible, in particularly simple manner, to dispense a plurality of needles with related suture thread ends at the same time, for example, from the needle dispensing region to the needle accommodation region.

In order to easily be able to displace the aforementioned elongated outer member on the rod-shaped needle and thread guidance device, in its longitudinal direction, the elongated outer member in question is connected with a pusher device provided within or outside of the rod-shaped needle and thread guidance device, and can be displaced relative to the rod-shaped needle and thread guidance device, in the longitudinal direction of the latter, by means of this device.

Using drawings, the invention will be explained in greater detail below, on the basis of an exemplary embodiment.

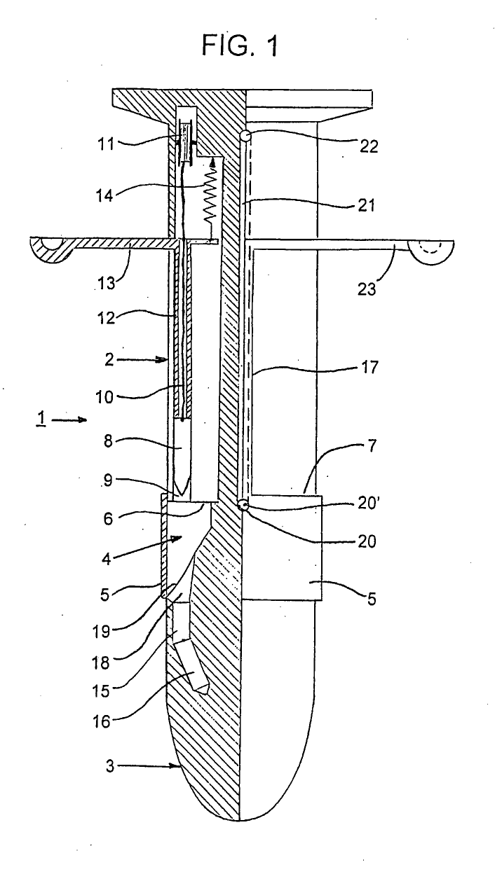

FIG. 1 shows, in a schematic half-section representation and on a greatly enlarged scale, a surgical device according to an embodiment of the present invention.

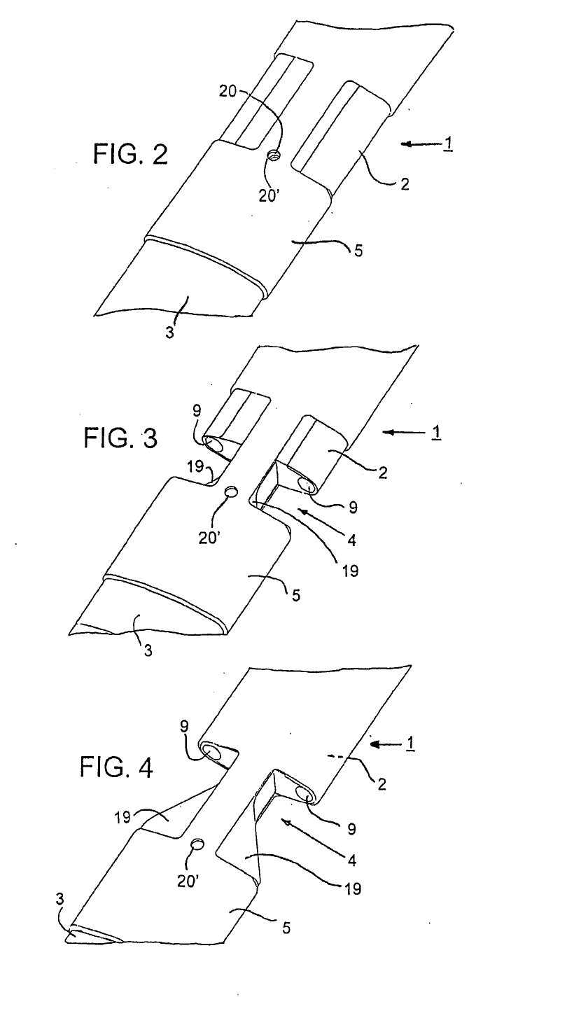

FIGS. 2, 3 and 4 show, in perspective details and also on greatly enlarged scales, a part of the surgical device shown in FIG. 1, in different working positions, in each instance.

The surgical device shown in FIG. 1 in a schematic half-section representation and on a greatly enlarged scale, according to an embodiment of the present invention, consists of a rod-shaped needle and thread guidance device 1, which contains a needle dispensing region 2 provided at the proximal end of the device, the part shown at the top in FIG. 1, a needle accommodation region 3 provided on the distal end of the device, the part shown at the bottom in FIG. 1, and an intermediate space region 4 provided between the needle dispensing region 2 and the needle accommodation region 3. These three regions 2, 3, and 4 of the device shown are rigidly connected with one another. It is true that in FIG. 1, these three regions 2, 3, and 4 are shown as consisting of one material piece, for example made of a plastic such as PEEK (polyether ether ketone), for example, or of metal, such as titanium or a titanium alloy; however, it is understood that the regions in question can also be made from individual parts, which are then firmly, i.e. rigidly connected with one another, such as by means of screw connections, for example. With regard to the aforementioned scale of the device shown, it should be noted here that the outside diameters of the needle dispensing region 2 and of the needle accommodation region 3 can amount to 3 mm to 4 mm, in each instance, in practice.

The intermediate space region 4 of the rod-shaped needle and thread guidance device 1, which will be discussed in greater detail below, is according to FIG. 1 surrounded, with shape fit, by an elongated outer member 5, which can be, for example, a thin sheet part, e.g. made of a plastic or metal, which rests firmly against the outside circumference of the rod-shaped needle and thread guidance device 1, but can be displaced in the longitudinal direction of the needle and thread guidance device 1 in question, something that will be discussed in greater detail below. As is evident from FIG. 1, the intermediate space region 4 can be completely closed off towards the outside of the rod-shaped needle and thread guidance device by means of the aforementioned elongated outer member 5.

The needle dispensing region 2 has an end side 6, here configured as a region configured with rotation symmetry, towards the distal end of the device, and the elongated outer member 5 also has an end side 7 in its region towards the proximal end of the device; these two end sides 6 and 7 face one another, essentially, and run parallel to one another; in the present case, they furthermore run at a right angle to the longitudinal direction of the needle and thread guidance device 1.

The needle dispensing region 2 is provided with a needle 8, in each instance, in its part shown in section and also in its part not shown in section, which needle is accommodated in a needle guidance opening 9 and with which the one end of a suture thread 10 is connected, which thread is supplied from a supply roller 11 provided at the proximal end of the device.

In FIG. 1, it is also indicated that a needle pusher 12 sits on the proximal end of the visible needle 8, which pusher can be activated by means of an activation lever 13, which is situated close to the proximal end of the device. A tension spring 14 holds the activation lever 13 and thereby the needle pusher 12 in the position indicated in FIG. 1, in which the needle 8 is situated in the needle dispensing region 2. The activation lever 13 in question can, of course, also be effective for a corresponding needle pusher and a corresponding needle in the region of the needle dispensing region 2 not shown in section.

The needle accommodation region 3 provided at the distal end of the device contains accommodation openings, such as the accommodation opening 15 shown in FIG. 1, which aligns with the related needle guidance opening 9 provided in the needle dispensing region 2, for the needles that can be dispensed from the needle dispensing region 2, such as the needle 8 shown. In FIG. 1, the needle accommodation 15 is furthermore connected with a bent-away sealing opening 16, which runs at a slight angle with regard to the accommodation opening 15. By means of this measure, it is assured here that the needle 8 introduced into the needle accommodation region 3 is securely clamped in the latter, and thereby permanently held tightly in the needle accommodation region 3. Towards the proximal end, the accommodation opening 15 is equipped with a funnel-shaped needle inlet region 18, which allows easier introduction of the needle 8 into the accommodation opening 15. This characteristic is particularly advantageous when using needles that are shorter than the needle 8 shown in FIG. 1.

The intermediate space region 4 provided between the needle dispensing region 2 and the needle accommodation region 3 has a length corresponding at least to the thickness of the wall of a tissue opening that is to be closed by means of the device in question, and an outer shape that is reduced with reference to the outer shape of the needle dispensing region 2 and/or of the needle accommodation region 3, for unhindered movement of the needles in question, such as the needle 8, between the needle regions 2, 3 in question. In FIG. 1, it is shown that the intermediate space region 4 in question is essentially constricted with regard to the needle dispensing region 2 and the needle accommodation region 3. Furthermore, the needle accommodation region 3 has a bevel 19 that runs from its outside circumference in the direction towards the center axis of the device, on its side facing the proximal end of the device. This bevel 19 therefore runs from the distal end of the device towards the proximal end of the device. The bevel 19 in question preferably has an angle of ≦45° with reference to the center axis of the device.

In the case of the device according to an embodiment of the invention shown in FIG. 1, the intermediate space region 4 is provided with a passage opening 20, close to the edge towards the needle dispensing region 2, which opening coincides here, at the same time, with a passage opening 20′ of the elongated outer member 5, which has the same coverage. In the present case, the passage opening 20 is provided with a connection opening 21 provided in the interior of the needle dispensing device 2, having an exit opening 22 provided at the proximal end of the device, which latter opening can be provided with a display device, such as a viewing glass, if necessary. The significance of the passage opening 20 and the connection opening 21 connected with it, as well as of the exit opening 22, will be discussed in further detail below.

With regard to the elongated outer member 5, it should still be noted here that according to FIG. 1, this outer member is connected with a crosspiece part 17 having an activation lever 23 indicated at the proximal end of the device, which can be used to displace the elongated outer member 5 in the longitudinal direction of the needle and thread guidance device 1, relative to the latter. In the present case, a displacement from the position shown in FIG. 1 takes place only towards the distal end of the device. For the remainder, the displacement of the elongated outer member 5 could also take place by means of a pushing device guided in the interior of the rod-shaped needle and thread guidance device 1, running towards the proximal end of the device and connected with the outer member 5 in question. Now that the fundamental structure of an embodiment of the surgical device according to the invention has been explained using the partial section image shown in FIG. 1, the special effects that are connected with the use of this device in practice will now be discussed, making reference to FIGS. 2, 3, and 4.

First of all, it should be noted, in general, making reference to FIG. 2 to 4, that there, a modified rod-shaped needle and thread guidance device 1 is shown as compared with the device shown in FIG. 1, with regard to the elongated outer member 5, having an oval cross-section, whereby the needle dispensing region 2 and the needle accommodation region 3 are disposed on the greater cross-section axis of the oval cross-section region in question only close to the end regions. This means that needle dispensing openings 9 according to FIG. 1 and related needle accommodation openings 15 according to FIG. 1 lie on the greater cross-section axis of the oval cross-section in question. This is clearly shown in FIGS. 3 and 4 with regard to the needle dispensing openings 9.

The perspective view shown in detail in FIG. 2 shows the surgical device according to an embodiment of the present invention corresponding to the position shown in FIG. 1, in other words in a position in which the intermediate space region 4 is completely closed off by the elongated outer member 5. From FIG. 2, it is furthermore evident that the passage opening 20 of the intermediate space region 4 mentioned in connection with FIG. 1 is in coverage with the passage opening 20′ of the elongated outer member. In the position shown in FIG. 2, the surgical device in question is introduced into a tissue opening to be closed, such as an artery, specifically to such an extent until the passage openings 20, 20′ are situated within the tissue opening in question, and therefore a corresponding tissue fluid can get through these passage openings and the connection opening 21 mentioned in connection with FIG. 1, to the exit opening 22. FIG. 3 shows the surgical device, details of which are shown in FIG. 2, in a perspective view, in a position in which the elongated outer member 5 is displaced towards the distal end of the device. Now the passage openings, of which now only the passage opening 20′ is visible in FIG. 3, are no longer in coverage with one another, but rather the intermediate space region 4 is slightly open. In this case, the edge region of the tissue opening to be closed can be accommodated by this intermediate space region 4, whereby the end sides that face one another, namely the end side 6 of the needle dispensing region 2 and the end side 7 of the elongated outer member 5 can essentially undertake fixation of the entire surgical device and thereby its precise and secure positioning on the edge region of the tissue opening to be closed. Back and forth movement of the entire surgical device, with the edge region of the tissue opening in question accommodated in its intermediate space region 4, furthermore does not have the result that the entire device slips out of the tissue opening in question. Instead, precise and secure positioning and fixation of the entire device on the edge region of the tissue opening to be closed is assured by means of the aforementioned end sides 6 and 7, which face one another.

In the position of the surgical device shown in FIG. 3, it is practical if the needles contained in the needle dispensing openings 9, such as the needle 8 shown in FIG. 1, are introduced into the needle accommodation region 3, together with the suture threads connected with them. The elongated outer member 5, which still surrounds part of the intermediate space region 4 in this position, makes a contribution, in this connection, that the needles can be securely introduced into their corresponding needle accommodation openings, such as the accommodation opening 15 according to FIG. 1. This needle guidance is furthermore supported by the funnel-shaped configuration of the needle introduction in the needle accommodation region 3 mentioned in connection with FIG. 1.

In the position of the surgical device according to the invention shown in FIG. 4, the elongated outer member 5 is displaced towards the distal end of the device so far that the intermediate space region 4 is completely exposed. In this position, the surgical device in question can be pulled out of the tissue opening without hindrance; previously, needles such as the needle 8 shown in FIG. 1, with corresponding suture threads, have been guided through the edge regions of the opening, and permanently accommodated in the needle accommodation region 3. The needles in question are clamped tightly in the needle accommodation region 3, in such a manner that even when the device in question is pulled back out of the tissue opening, the needles continue to remain in the needle accommodation region 3, but the suture threads connected with them can be pulled back out of the aforementioned tissue opening, so that these suture thread ends and the other suture thread ends of the suture threads in question, in each instance, can be knotted together outside of the tissue opening in question, in order to close this tissue opening.

With regard to the conditions shown in FIGS. 2, 3, and 4, it can therefore be stated, in summary, that FIG. 2 shows the surgical device in an introduction position during introduction into a tissue opening to be closed, through the edge region of which at least two suture threads are to be guided; FIG. 3 shows the surgical device in question in a position in which its needles, with the suture threads that are connected with them, are being guided through the edge region of the tissue opening to be closed; FIG. 4, finally, shows the device in question in the position in which it can be completely pulled out of the tissue opening in question, i.e. is being pulled out. In this connection, the edge region of the tissue opening in question has two suture threads pulled through it here, the ends of which can be pulled together and knotted outside of the tissue opening in question, closing it.

In conclusion, it should still be noted that in FIG. 2 to 4, the rod-shaped needle and thread guidance device 1 is shown as having an oval cross-section. According to the invention, however, it is also possible that the needle and thread guidance device 1 in question has a circular cross-section, or a cross-section that approximates the latter, and that the needle dispensing region 2 and the needle accommodation region 3 are disposed distributed around the cross-section in question, with a corresponding plurality of needle dispensing openings and needle accommodation openings, respectively (with n≦2).

### REFERENCE SYMBOL LIST

1 needle and thread guidance device
2 needle dispensing region
3 needle accommodation region
4 intermediate space region
5 elongated outer member
6 end side
7 end side
8 needle
9 needle guidance opening
10 suture thread
11 suture thread
12 needle pusher
13 activation lever
14 tension spring
15 accommodation opening
16 sealing opening
17 crosspiece part
18 needle inlet region
19 bevel
20 passage opening
20′ passage opening
21 connection opening
22 exit opening
23 activation lever

1. Surgical device for guiding at least two suture threads through the edge region of a tissue opening, particularly one present in the artery of an individual, if applicable formed by an incision, and for retracting the suture thread ends guided through the edge region in question, in each instance, from the stated opening, having a rod-shaped needle and thread guidance device, in which suture threads attached to needles, in each instance, are guided in guidance and/or accommodation openings, in such a manner that these can be moved from a needle dispensing region disposed at the proximal end of the device to a needle accommodation region at the distal end of the device, by way of an intermediate space region, whereby the intermediate space region in question has a length that at least corresponds to the thickness of the wall of the tissue opening, and an outer shape, with reference to the outer shape of the needle dispensing region and/or the needle accommodation region, that is reduced for unhindered movement of the needles in question between the needle regions in question, whereby the stated needles can be moved to the needle accommodation region, for permanent accommodation there, after having been guided through the edge region of the tissue opening in question, and whereby afterwards, the rod-shaped needle and thread guidance device, together with the needles permanently accommodated in the needle accommodation region, and the suture thread ends connected with them, can be retracted from the stated tissue opening again, in such a manner that the latter can be closed by means of drawing together all of the suture thread ends, outside of the tissue opening in question,
wherein the needle dispensing region (2), the intermediate space region (4), and the needle accommodation region (3) of the rod-shaped needle and thread guidance device (1) are rigidly connected with one another,
that the rod-shaped needle and thread guidance device (1) can be closed off by an elongated outer member (5) that surrounds the intermediate space region (4) with shape fit, which member can be displaced relative to the rod-shaped needle and thread guidance device (1), in its longitudinal direction, releasing the stated intermediate space region (4),
and that the needle dispensing region (2) has an end side (6) towards the distal end of the device, and the elongated outer member has an end side (7) in its region towards the proximal end of the device, which end sides (6, 7) face one another.

2. Device according to claim 1,
wherein the end sides (6, 7) in question run parallel to one another, or at a right angle to the longitudinal direction of the rod-shaped needle and thread guidance device (1).

3. Device according to claim 1,
wherein the needle accommodation openings (15) of the needle accommodation region (3) are configured in funnel shape in their needle inlet region (18), in each instance.

4. Device according to claim 1,
wherein the needle accommodation region (3) has a bevel (19) that runs from its outside circumference in the direction towards the center axis of the device, on its side facing the proximal end of the device.

5. Device according to claim 4,
wherein the bevel (19) forms an angle of ≦45° with reference to the center axis of the device.

6. Device according to claim 1,
wherein the rod-shaped needle and thread guidance device (1) has a passage opening (20) in its intermediate space region (4), which opening is connected with a connection opening (21) present in the rod-shaped needle and thread guidance device (1) that runs towards its proximal end.

7. Device according to claim 6,
wherein the elongated outer member (5) also has a passage opening (20′), which is connected with the passage opening (20) situated in the intermediate space region (4) of the rod-shaped needle and thread guidance device (1) in the position of the elongated outer member (5) that closes off the aforementioned intermediate space region (4) of the rod-shaped needle and thread guidance device (1).

8. Device according to claim 1,
wherein the rod-shaped needle and thread guidance device (1) has an oval cross-section, and that the needle dispensing region (2) and the needle accommodation region (3) are disposed close to the end regions only on the larger cross-section axis of the oval-shaped cross-section in question.

9. Device according to claim 1,
wherein the rod-shaped needle and thread guidance device (1) has a circular cross-section or a cross-section that approximates the latter, and that the needle dispensing region with corresponding needle guidance openings and the needle accommodation region with corresponding needle accommodation openings are disposed distributed around the cross-section in question.

10. Device according to claim 1,
wherein the elongated outer member (5) is connected with a pusher device (17, 23) provided within or outside of the needle and thread guidance device (1), and can be displaced in the longitudinal direction of the needle and thread guidance device (1), from the proximal end of the device, by means of this device.
